(19) United States
(12) Patent Application Publication
(10) Pub. No.: US 2018/0228546 A1
(43) Pub. Date: Aug. 16, 2018

(54) THERMAL TREATMENT PLANNING SYSTEM AND THERMAL TREATMENT PLANNING METHOD

(71) Applicant: Metal Industries Research & Development Centre, Kaohsiung (TW)

(72) Inventors: Yu-Jie Lan, Kaohsiung City (TW); Tung-Chieh Yang, Pingtung County (TW); Tsung-Chih Yu, Tainan City (TW)

(73) Assignee: Metal Industries Research & Development Centre, Kaohsiung (TW)

(21) Appl. No.: 15/828,346

(22) Filed: Nov. 30, 2017

(30) Foreign Application Priority Data
Feb. 10, 2017 (TW) 106104432

(51) Int. Cl. A61B 34/10; A61B 34/00; A61B 90/00

(52) U.S. Cl. CPC A61B 34/10; A61B 2018/00089; A61B 90/37; A61B 34/25

(57) ABSTRACT

A thermal treatment planning system including a processing device and an image display device is provided. The processing device obtains a medical condition setting, and performs a curve fitting operation according to a plurality of surgical situation information provided by a surgical parameters database and the medical condition setting, so as to obtain a surgical evaluation information corresponding to the medical condition setting. The image display device displays the surgical evaluation information. If the surgical evaluation information is reliable, the processing device optimizes the plurality of surgical context information in the surgical situation database based on the surgical evaluation information. In addition, a thermal treatment planning method is also provided.

### CROSS-REFERENCE TO RELATED APPLICATION

This application claims the priority benefit of Taiwan application serial no. 106104432, filed on Feb. 10, 2017. The entirety of the above-mentioned patent application is hereby incorporated by reference herein and made a part of this specification.

### BACKGROUND OF THE INVENTION

### 1. Field of the Invention

The invention relates to a surgery planning technique, and particularly relates to a thermal treatment planning system and a thermal treatment planning method.

### 2. Description of Related Art

Cancers (or tumors) are one of the common severe diseases of human beings, and are also found to be one of the top-three causes of death according to the statistics of multiple countries. Thus, cancer treatments are desperately called for in the healthcare systems among countries, and the development of medical devices in connection with cancer treatment is also a major research area. Meanwhile, thermal treatment surgeries for tumors are one of the conventional cancer treatment techniques. Examples of such surgeries include thermal magnetic tumor ablation, such as radio frequency ablation (RFA) and microwave ablation (MWA). These techniques are already applied in some tumor treatments. However, considering the cost and limitations, these techniques are rarely adopted for most of the patients.

Through the rapid development of thermal treatment surgery, thermal treatment surgeries for tumors have become one of the solutions to completely cure tissue proliferation, benign tumors, and malignant tumors. For a desirable clinical effect, surgery planning, precise positioning during surgery, and post-surgery evaluation are indispensable key steps throughout the course of thermal treatment. As the first step of the whole treatment, the purpose of surgery planning is to ensure a safe treatment range of the thermal treatment surgery under the premise of reducing complications, so as to facilitate the treatment quality for the patient. The quality of surgery planning not only has a direct impact on post-surgery recovery, but is also an important aspect in ensuring the safety and precision of the surgery. A precise surgery plan is particularly important when the thermal treatment surgery is performed on tissues of an organ with important nerves and a large number of blood vessels. Besides, different patients or different body parts may exhibit different physical properties of thermal conduction. Hence, the importance of surgery planning is beyond question. In view of the above, how to develop a thermal treatment surgery planning system capable of providing effective surgical evaluation information is now an important issue for thermal treatment surgery.

### SUMMARY OF THE INVENTION

The invention provides a thermal treatment planning system and a thermal treatment planning method capable of carrying out simulation based on an input medical condition setting and a surgical situation database, so as to obtain a corresponding surgical situation information as a surgical evaluation information. In addition, the surgical evaluation information may be displayed via an image display device. Besides, the thermal treatment planning system and the thermal treatment planning method according to the embodiments may further determine whether to optimize the plurality of surgical situation information in the surgical situation database based on the surgical evaluation information according to whether the surgical evaluation information is reliable.

A thermal treatment planning system according to an embodiment of the invention includes a processing device and an image display device. The processing device is configured to obtain a medical condition setting, and perform a curve fitting operation according to a plurality of surgical situation information provided by a surgical parameters database and the medical condition setting, so as to obtain a surgical evaluation information corresponding to the medical condition setting. The image display device is coupled to the processing device. The image display device is configured to display the surgical evaluation information. If the surgical evaluation information is reliable, the processing device optimizes the plurality of surgical situation information in the surgical context database based on the surgical evaluation information.

A thermal treatment planning method according to an embodiment of the invention is adapted for a thermal treatment planning system. The thermal treatment planning system includes a processing device and an image display device. The thermal planning method includes steps as follows: obtaining a medical condition setting; performing a curve fitting operation based on a plurality of surgical situation information provided by a surgical situation database and a plurality of setting parameters included in the medical condition setting, so as to obtain a surgical evaluation information corresponding to the medical condition setting; and displaying the surgical evaluation information and determining whether to optimize the plurality of surgical situation information in the surgical situation database based on the surgical evaluation information according to whether the surgical evaluation information is reliable.

Based on the above, in the thermal treatment planning system and the thermal treatment planning method according to the embodiments of the invention, when the thermal treatment planning system receives the medical condition setting, the thermal treatment planning system may obtain the corresponding surgical situation information from the surgical situation database according to the setting parameters provided in the medical condition setting, so as to provide the surgical evaluation information. In other words, the surgical treatment planning system according to the embodiments of the invention may provide customized professional surgical plans for different patients based on different needs for thermal treatment surgery.

In order to make the aforementioned and other features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic view illustrating a thermal treatment planning system according to an embodiment of the invention.

FIG. 2 is a schematic view illustrating a thermal treatment planning system according to another embodiment of the invention.

FIG. 3 is a flowchart illustrating a thermal treatment planning method according to an embodiment of the invention.

FIGS. 4A and 4B are schematic views illustrating determining whether surgical evaluation information is reliable according to an embodiment of the invention.

FIG. 5 is a flowchart illustrating measuring, analyzing, and validating a tissue thermal property parameter according to an embodiment of the invention.

FIG. 6 is a flowchart illustrating a thermal treatment planning method according to another embodiment of the invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

A thermal treatment planning system according to the embodiments of the invention is capable of analyzing a medical image (such as an image obtained through computed tomography (CT), magnetic resonance imaging (MRI), or ultrasonic waves) of a tissue of a biological organ, a physical property parameter of a needling instrument or a biological tissue, and an electrical property parameter. The thermal treatment planning system is further capable of simulating a plurality of surgical situations to set up a surgical situation database. Accordingly, different surgical plans may be provided based on different physical properties of thermal conduction of tissues at different body parts or of different patients. In the following, several embodiments meeting the principle of the invention are provided. Those adopting the embodiments may properly adjust the embodiments based on needs and the invention is not limited to the descriptions in the following.

FIG. 1 is a schematic view illustrating a thermal treatment planning system according to an embodiment of the invention. Referring to FIG. 1, a thermal treatment planning system 100 includes a processing device 110 and an image display device 120. In the embodiment, the processing device 110 may include a surgical parameter database 111. In addition, based on a plurality of surgical parameters stored in the surgical parameter database 111, the processing device 110 may simulate a plurality of surgical situation information and set up a surgical situation database 113 based on the surgical situation information. Therefore, when the processing device 110 receives a medical condition setting MS from an external source, the processing device 110 may perform a simulated operation based on a plurality of setting parameters in the medical condition setting MS, so as to compare whether a surgical situation information corresponding to the medical condition setting MS is available in the surgical situation database 113. Besides, in the embodiment, after displaying a surgical evaluation information, the thermal treatment planning system 100 may receive a confirmation signal from the external source to determine whether surgical evaluation information is reliable. If the surgical evaluation information is unreliable, the image display device 120 may display a request to input the medical condition setting again. Alternatively, if the surgical evaluation information is reliable, the processing device 110 may feed the surgical situation information corresponding to the surgical evaluation information to the surgical situation database 113, so as to optimize the surgical situation database 113. It should be noted that medical staff may refer to the image display device 120 for a surgical evaluation result and a specific thermal treatment condition, so as to determine whether the surgical evaluation information is reliable.

In the embodiment, when the setting parameters of the medical condition setting MS match the corresponding surgical situation information SI, the processing device 110 may adopt the corresponding surgical situation information SI as the surgical evaluation information. In addition, the processing device 110 may provide the corresponding surgical situation information SI to the image display device 120, so that the image display device 120 may display the surgical evaluation information based on the corresponding surgical situation information SI. However, when the corresponding surgical situation matching the setting parameters of the medical condition setting MS is not available in the surgical situation database, the processing device 110 may perform a curve fitting operation based on the setting parameters of the medical condition setting MS and the plurality of surgical situation information in the surgical situation database, so as to find a corresponding surgical situation information close to the medical condition setting MS.

In the embodiment, the surgical evaluation information refers to an evaluation information about a surgery obtained through simulation. In addition, the surgical evaluation information may at least include a temperature distribution information and a thermal damage range information. In other words, when operating the thermal treatment planning system 100 of the embodiment, the medical staff may input the medical condition setting MS to the thermal treatment planning system 100 and be informed of relevant surgical evaluation information through the image display device 120.

In the embodiment, the processing device 110 may be an electronic apparatus having a computing capability, such as a desktop computer, a laptop computer, a server, a workstation computer, or the like. The processing device 110 may at least include a processor and a storage. The processor may be a central processing unit (CPU), a microprocessor, a digital signal processor (DSP), a programmable controller, an application specific integrated circuit (ASIC), a system on chip (SoC), other similar components, or a combination thereof, for example. The storage may be any type of static or portable random access memory (RAM), read-only memory (ROM), flash memory, similar components, or a combination thereof, for example.

In the embodiment, the image display device 120 may be a liquid crystal display (LCD), a thin film transistor liquid crystal display (TFT-LCD), a light emitting diode (LED) display, an organic light emitting diode (OLED) display, or the like, for example. In the embodiment, the processing device 110 may present the surgical evaluation information corresponding to the medical condition setting MS input by the medical staff, such as a surgical simulation information, a physical parameter, a thermal physical parameter, a temperature distribution, and a thermal damage condition information, through the image display device 120.

In addition, the curve fitting operation of the embodiment refers to an empirical formula, which is a statistical method adopted in the fields of engineering, science and economics. Specifically, the curve fitting operation of the embodiment is carried out by performing computation on a plurality of dispersed data values obtained through experimentation or sampling based on a continuous function (i.e., a function obtained according to a plurality of surgical parameters) and finding an optimal match function for these values (i.e., obtaining the corresponding surgical situation information) through calculating a minimum sum of squared errors. Sufficient teaching, suggestions, and descriptions for implementing the detailed calculation and technical feature of the curve fitting operation are readily available in the common knowledge of the related technical field. Thus, details in this regard will not be repeated in the following.

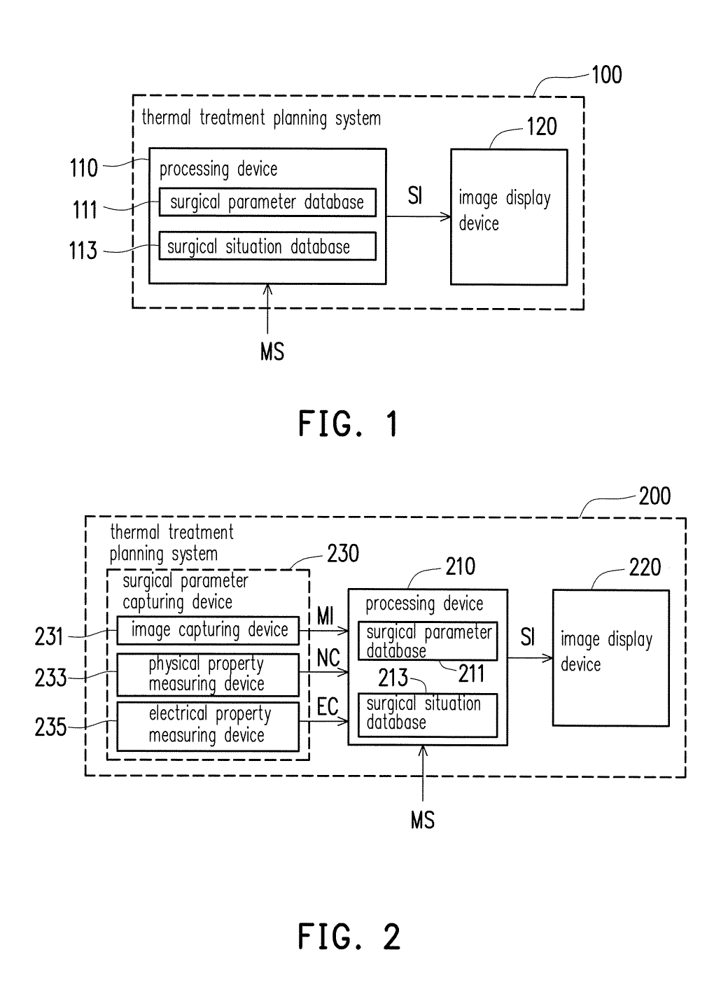

FIG. 2 is a schematic view illustrating a thermal treatment planning system according to another embodiment of the invention. Referring to FIG. 2, a thermal treatment planning system 200 includes a processing device 210 and an image display device 220. Compared with the embodiment of FIG. 1, the thermal treatment planning system 200 of the embodiment may further include a surgical parameter capturing device 230. The surgical parameter capturing device 230 is configured to obtain a plurality of surgical parameters. For example, the surgical parameters may include property parameters relating to a tissue of a biological organ or a needling instrument, such as a tissue profile parameter, a tissue thermal property parameter, a physical property parameter of a needing instrument or a biological tissue, an electrical property parameter, and the like. The tissue thermal property parameter may be inferred from medical experimentation or relevant literature and stored in advance in the surgical parameter database 211.

In the embodiment, the surgical parameter capturing device 230 may include an image capturing device 231, a physical property measuring device 233, and an electrical property measuring device 235. The image capturing device 231 may be configured to capture a medical image MI of a tissue of a biological organ and provide the medical image MI to the processing device 210. Accordingly, the processing device 210 may analyze the medical image MI to obtain the relevant tissue profile parameter. The physical property measuring device 233 may be configured to measure a needling instrument or a biological issue of the thermal treatment surgery to obtain a relevant physical property parameter NC and provide the physical property parameter NC to the processing device 210. For example, the physical property measuring device 233 may be configured to measure a temperature, a length, and a diameter of the needling instrument or a parameter about the tissue thermal property of the biological tissue. However, the invention is not limited thereto. The electrical property measuring device 235 may be configured to measure the needling instrument used in the thermal treatment surgery to obtain an electrical property parameter EC and provide the electrical property parameter EC to the processing device 210. For example, the electrical property measuring device 235 may be configured to measure an electrical magnetic field intensity, an operating frequency, or a current amplitude. However, the invention is not limited thereto.

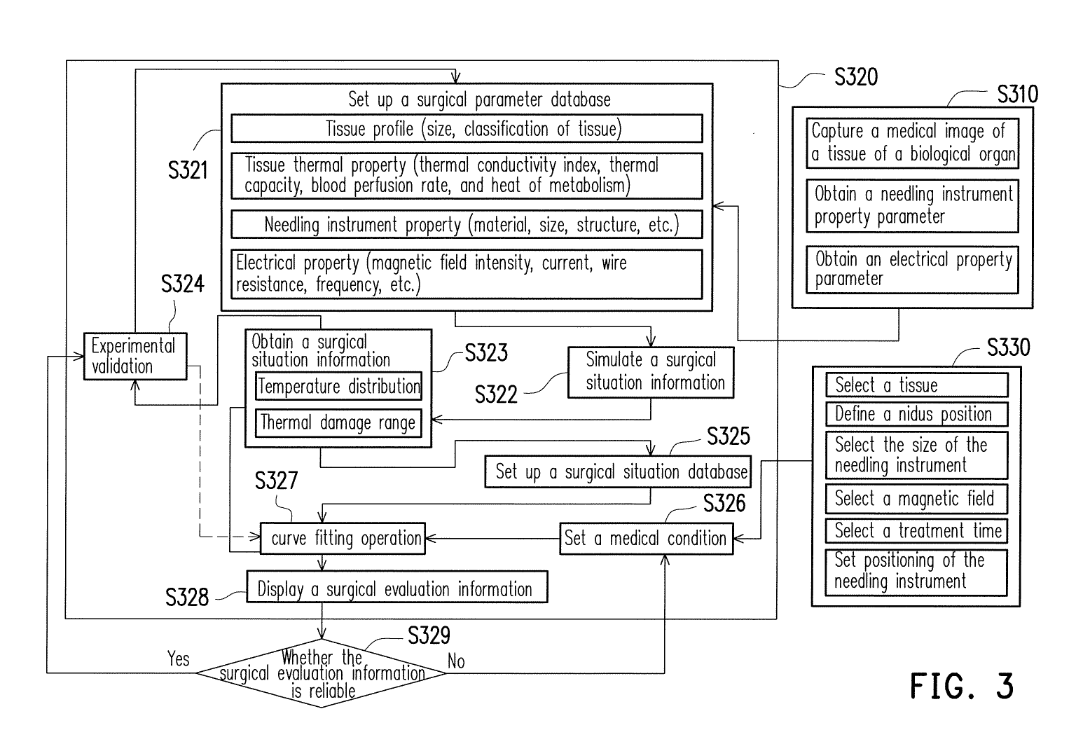

Specifically, FIG. 3 is a flowchart illustrating a thermal treatment planning method according to an embodiment of the invention. Referring to FIGS. 2 and 3, in the thermal treatment planning system 200, the surgery parameter capturing device 230 may be configured to capture the surgical parameters of the tissue of the biological organ (Step S310) and provide the medical image MI, the physical property parameter NC and the electrical property parameter EC of the needling instrument or the tissue of the biological organ that are obtained to the processing device 210. In the embodiment, the physical property parameter NC includes a material property, a size, and a structure of the needling instrument, for example, or a thermal physical parameter of the tissue of the biological organ. The electrical property parameter EC includes a magnetic field intensity, a current, a wire resistance and a frequency of the needling instrument, for example. Then, based on the surgical parameters provided by the surgery parameter capturing device 230, the processing device 210 sets up the surgical parameter database 211 (Step S321) and simulates various surgical situations based on the surgical parameter database 211 to obtain the plurality of surgical situation information and set up a surgical situation database 213 (Step S325). Accordingly, the medical staff may input the medical condition setting through an expert system interface of the thermal treatment planning system 200 (Step S330). The medical condition setting may include relevant setting parameters such as selection of a tissue, definition of a nidus position, selection of the size of the needling instrument, selection of treatment time, and positioning setting of the needling instrument. In other words, the processing device 210 may obtain the corresponding surgical situation information SI based on the medical condition MS input by the medical staff, so that the image display device 220 may display the surgical evaluation information based on the corresponding surgical situation information SI.

Specifically, the processing device 210 may analyze the medical image MI of the tissue of the biological organ (Step S321) to obtain the relevant tissue profile parameter. The tissue profile parameter includes a tissue size or tissue classification of the tissue of the biological organ, for example. The tissue thermal property parameter includes a thermal conductivity index, a thermal capacity, a blood perfusion rate, and a heat of metabolism of the tissue of the biological organ, for example. Then, the processing device 210 simulates the surgical situation information based on the respective surgical parameters (Step S322) to obtain a plurality of surgical situation information (Step S323). The surgical situation information may include a temperature distribution information and a thermal damage range information during the thermal treatment surgery. Besides, the processing device 210 may perform experimental validation on a simulation result (Step S324) to determine whether the surgical situation information in the simulation result is correct. In other words, the processing device 210 may determine whether the surgical situation information is correct based on actual experimental errors of the respective surgical parameters, so as to determine whether to adjust the surgical parameters in the surgical parameter database 221. Then, the processing device 210 stores the validated surgical situation information and sets up the surgical situation database 213 (Step S325). Thus, when the processing device 210 receives the medical condition setting MS input from the external source (Step S326), the processing device 210 may perform the curve fitting operation based on the setting parameters provided in the medical condition setting MS and the plurality of surgical situation information stored in the surgical parameter database 213 (Step S327). Then, the processing device 210 may output the corresponding surgical situation information SI obtained through simulation to the image display device 220 (Step S328). Accordingly, the image display device 220 may display the surgical evaluation information. Besides, in the curve fitting operation of the embodiment, an operation parameter in the curve fitting operation may also be modified through experimental validation, so as to modify an operation result of the surgical situation information obtained through the curve fitting operation. Then, the medical staff may input a confirmation signal from the external source to the processing device 210 to determine whether the surgical evaluation information is reliable (Step S329). If the medical staff determines that the surgical evaluation information is not reliable, the image display device 220 may display the request to input the medical condition setting MS again. Alternatively, if the medical staff determines that the surgical evaluation information is reliable, the processing device 210 may feed the surgical situation information SI corresponding to the surgical evaluation information to the surgical situation database 113, so as to optimize the surgical situation database 113.

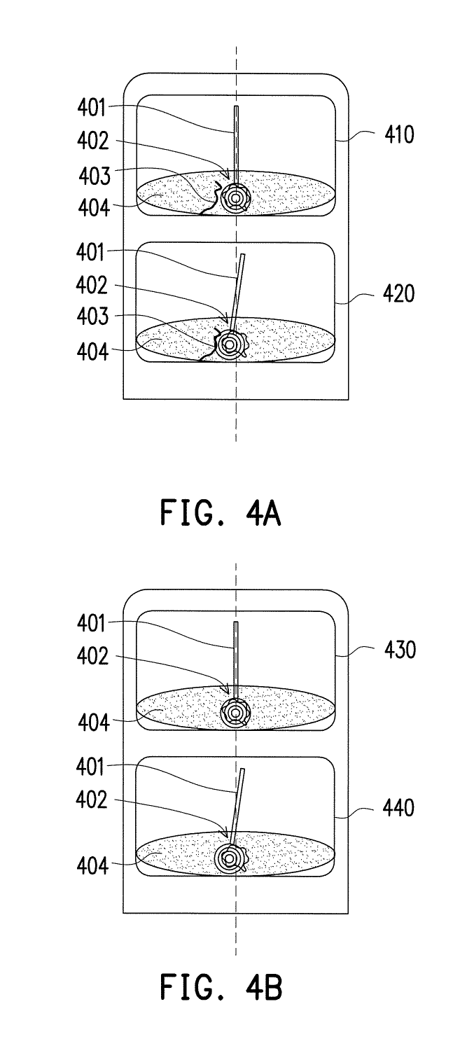

FIGS. 4A and 4B are schematic views illustrating determining whether surgical evaluation information is reliable according to an embodiment of the invention. Referring to FIG. 4A, in the embodiment, the surgical evaluation information may present a simulated image 410 as shown in FIG. 4A, for example. The simulated image 410 may show that a needling instrument 401 is placed at a nidus position of a tissue 404 of a biological organ and show a thermal treatment range 402. In the embodiment of FIG. 4A, the nidus position may be near a nervous tissue 403. Thus, in an actual image 420 of needle insertion, there may be an error in a position where the needling instrument 401 is placed that may result in ablation at the nervous tissue 403. In other words, the medical staff may make comparison and evaluation based on the simulated image 410 and the actual needle insertion. If the thermal treatment is not plausible, the medical staff may determine whether to position a position of needle insertion again. Besides, when the thermal treatment surgery is actually performed, the medical equipment may include the needling instrument 401 and a positioning device (not shown). For example, positioning may be carried out by an infrared or microwave technique. Nevertheless, the invention is not limited thereto.

Besides, in another embodiment, a simulated image 430 in FIG. 4B may also be presented in the surgical evaluation information, for example. The simulated image 430 may show that the needling instrument 401 is placed at the nidus position of the tissue 404 of the biological organ and show the thermal treatment range 402. In the embodiment of FIG. 4B, if the nidus position is not adjacent to a nervous tissue, the medical staff may determine that the thermal treatment is plausible, and may determine whether to optimize the parameter setting, such as increasing a current conducted via the needling instrument or extending the thermal treatment time, based on the simulated image 430. However, in an actual image 440 of needle insertion, there may still be an error in the position where the needling instrument 401 is placed. Therefore, the medical staff may determine whether to insert the needle again based on a positioning result of the actual needle provided by the positioning device.

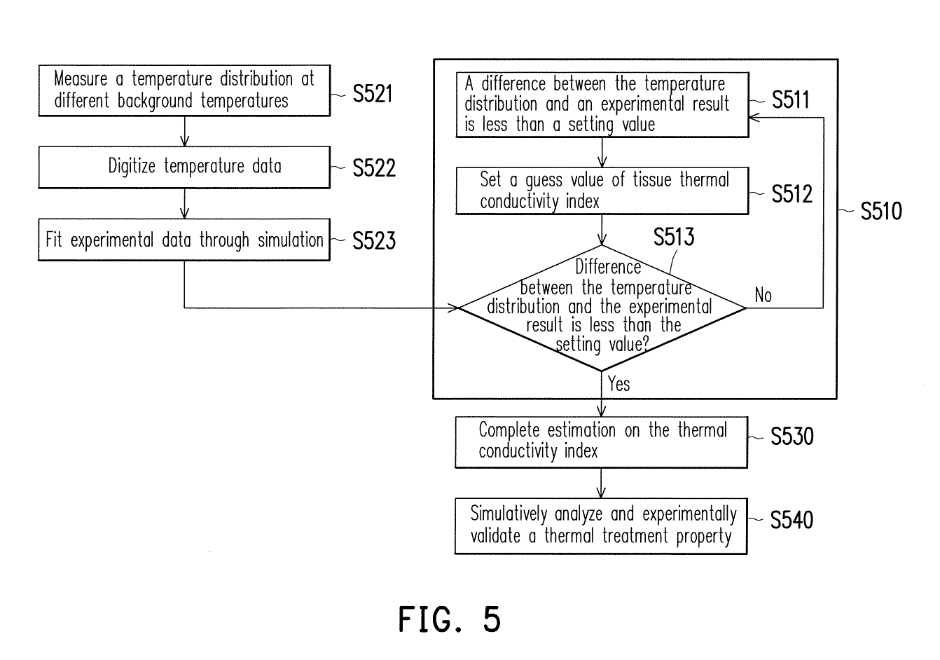

FIG. 5 is a flowchart illustrating measuring, analyzing, and validating a tissue thermal property parameter according to an embodiment of the invention. Referring to FIG. 5, the process is about measuring, analyzing, and validating the tissue thermal property. At Step S510, the processing device 210 executes a simulation program. First of all, the processing device 210 determines that a difference between the temperature distribution and an experimental result is less than a setting value. Then, the processing device 210 sets a guess value of the tissue thermal conductivity index (Step S512) and calculate a tissue temperature distribution by the simulation program. A value analysis program (e.g., the finite element method, the finite difference method, or the boundary element method) is set up to seek for a solution in a thermal conduction equation (e.g., the Pennes thermal conduction equation). Then, whether the difference between the temperature distribution and the experimental result is less than the setting value is determined (Step S513). Regarding generation of experimental data, the processing device 210 may firstly obtain temperature distributions at different background temperatures (Step S521), digitize temperature data, and fitting experimental data through simulation (Step S523) to generate the experimental data for determination at Step S513.

In the embodiment, if the difference between the temperature distribution and the experimental result is not less than (e.g., greater than or equal to) the setting value, Step S511 is performed again to reset the guess value of the tissue thermal conductivity index. In other words, the simulation program executed by the processing device 210 may perform iterative operation (i.e., repeating Step S511 to S513) when the difference between the temperature distribution and the experimental result is not less than the setting value. Alternatively, if the difference between the temperature distribution and the experimental result is less than the setting value, estimation of the thermal conductivity index is completed (Step S530). Then, the processing device 510 carries out a simulation analysis and an experimental validation on thermal treatment property (Step S540). For example, a thermal electric couple temperature, a thermal imager distribution or a thermal treatment range may be validated through experimentation.

However, the measurement, analysis, and validation of the tissue thermal property described in FIG. 5 are only an example, and the invention is not limited thereto. In an embodiment, the processing device 210 may perform experimental validation on the tissue profile parameter, the tissue thermal property parameter, and the physical property parameter and the electrical property parameter of the needling instrument or the biological tissue respectively.

In the embodiment, after the surgical situation database 213 is set up, the processing device 210 may receive the medical condition setting MS through the surgical parameter capturing device 230, and provide the corresponding temperature distribution and the thermal damage condition information based on the medical condition setting MS and the surgical situation database 213. In actual application, taking magnetic sensing thermal treatment as an example, the surgical parameter capturing device 230 may capture surgical operation parameters, such as a coil current, an alternate frequency, a needle diameter, a needle depth, and time input by the medical staff. Besides, based on different needs, the surgical situation database 213 may also be applicable in other types of thermal treatment. The invention does not intend to impose a limitation on this regard. Based on the medical condition setting MS and the surgical situation database 213, the processing device 210 may able to present the surgical evaluation information relating to the temperature distribution information and the thermal damage range information, such as the temperature distribution, the thermal treatment range, the tissue thermal damage range, the difference between the tissue thermal damage range and the expected treatment range given the surgical operation parameters.

For example, the processing device 210 may simulate the treatment range based on the surgical parameters such as the position of needle insertion, the number of needling instrument, and the like, to display a simulated treatment range and the actual medical image through the image display device 220. Hence, the medical staff is able to make observation, analysis and comparison from a plurality of perspectives and at a plurality of levels before the surgery.

Accordingly, in an evaluation procedure before the surgery, the thermal treatment planning system 200 according to the embodiment is able to assist the medical staff to gain a more straightforward understanding to the treatment situation under respective operation conditions. Besides, a spatial relationship between a treatment region and important structures nearby may also be obtained, thereby avoiding a thermal damage to the structures nearby is avoided. Hence, a diagnosis accuracy and a surgical quality and effect are able to be facilitated.

In another embodiment, during the surgery, the processing device 210 may further provide a surgical guidance information through the image display device 220. The surgical guidance information may include the temperature distribution and the thermal damage condition information. For example, the thermal treatment planning system 200 may include another image capturing device (not shown) to capture a surgical image. The processing device 210 may evaluate the respective operation conditions based on the surgical image and the surgical situation database 213, and present the corresponding surgical guidance information through the image display device 220, so as to simulate the respective processes and unexpected incidents during the surgery. Hence, the surgical guidance information is able to guide the medical staff to perform a corresponding operation or make correction.

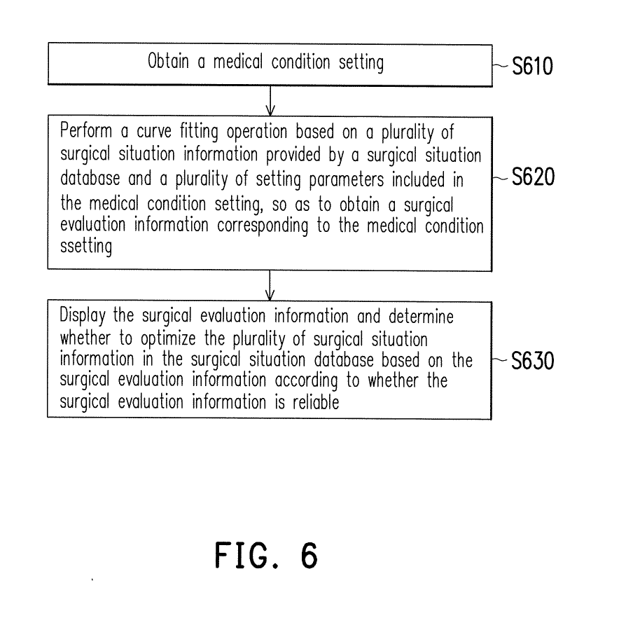

FIG. 6 is a flowchart illustrating a thermal treatment planning method according to another embodiment of the invention. Referring to FIGS. 1 and 6, the thermal treatment planning method according to the embodiment is at least suitable for the thermal treatment planning system 100 of FIG. 1. In the embodiment, the thermal treatment planning system 100 includes the processing device 110 and the image display device 120. At Step S610, the processing device 100 may obtain the medical condition setting. Then, at Step S620, the processing device 110 may perform the curve fitting operation based on the plurality of surgical situation information provided by the surgical situation database and the plurality of setting parameters included in the medical condition setting MS, so as to obtain the surgical evaluation information corresponding to the medical condition setting MS. Then, at Step S630, the image display device 120 may display the surgical evaluation information. In addition, the processing device 110 may determine whether to optimize the plurality of surgical situation information in the surgical situation database 113 based on the surgical evaluation information according to whether the surgical evaluation information is reliable. Hence, the thermal treatment planning method of the embodiment is able to provide customized surgical evaluation information for different patients based on different needs for thermal treatment surgery.

In view of the foregoing, in the thermal treatment planning system and the thermal treatment planning method according to the embodiments of the invention, the medical condition setting may be received so that the thermal treatment planning system may provide the corresponding surgical evaluation information. Hence, before the surgery, the thermal treatment planning system according to the embodiments of the invention may assist the medical staff and the patient to gain understanding to the surgery and further provide the surgical guidance information during the surgery. Hence, as the surgical planning system is formed by combining the medical image, the tissue thermal property database, the thermal treatment surgery database, the temperature distribution, and the thermal damage range information, the system is able to provide the surgical evaluation information obtained through simulation to the medical staff for comparison with the actual medical image, so as to gain understanding to the treatment condition and the spatial relationship between the treatment region and the nearby important structures. Thus, the chance of causing thermal damage to the structures nearby is reduced. Besides, after displaying the surgical evaluation information, the thermal treatment planning system according to the embodiments of the invention may receive the confirmation signal from the external source to determine whether the surgical evaluation information is reliable, and consequently determine whether to input the medical condition setting again or feed the surgical evaluation information back to the surgical situation database. Accordingly, the thermal treatment planning system and the thermal treatment planning method according to the embodiments of the invention are able to facilitate the diagnosis accuracy as well as the quality and effect of the surgery.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A thermal treatment planning system, comprising:
a processing device, configured to obtain a medical condition setting and perform a curve fitting operation based on a plurality of surgical situation information provided by a surgical situation database and a plurality of setting parameters comprised in the medical condition setting, so as to obtain a surgical evaluation information corresponding to the medical condition setting; and
an image display device, coupled to the processing device, and configured to display the surgical evaluation information,
wherein if the surgical evaluation information is reliable, the processing device optimizes the plurality of surgical situation information in the surgical situation database based on the surgical evaluation information.

2. The thermal treatment planning system as claimed in claim 1, wherein the thermal treatment planning system receives a confirmation signal from an external source to determine whether the surgical evaluation information is reliable,
if the surgical evaluation information is not reliable, the image display device displays a request to input the medical condition setting again, and
if the surgical evaluation information is reliable, the processing device feeds the surgical situation information corresponding to the surgical evaluation information to the surgical situation database to optimize the surgical situation database.

3. The thermal treatment planning system as claimed in claim 1, wherein the surgical evaluation information displayed by the image display device comprises a temperature distribution information and a thermal damage range information.

4. The thermal treatment planning system as claimed in claim 1, wherein the processing device further validates the plurality of surgical situation information based on a plurality of experimental validation results to determine whether to correspondingly adjust the plurality of surgical situation information.

5. The thermal treatment planning system as claimed in claim 4, wherein the processing device further adjusts the curve fitting operation based on the experimental validation results.

6. The thermal treatment planning system as claimed in claim 1, further comprising:
a surgical parameter capturing device, coupled to the processing device and configured to obtain a plurality of surgical parameters comprising at least one of a tissue profile parameter, a tissue thermal property parameter, a physical parameter of a needling instrument or a biological tissue and an electrical property parameter, wherein the processing device sets up the surgical parameter database based on the surgical parameters.

7. The thermal treatment planning system as claimed in claim 6, wherein the surgical parameter capturing device comprises:
an image capturing device, configured to capture a medical image of a tissue of a biological organ and provide the medical image to the processing device, so that the processing device to analyze the medical image and thereby obtain the tissue profile parameter.

8. The thermal treatment planning system as claimed in claim 6, wherein the surgical parameter capturing device comprises:
a physical property measuring device, configured to measure a needling instrument to obtain the property parameter of the needling instrument.

9. The thermal treatment planning system as claimed in claim 6, wherein the surgical parameter capturing device comprises:
an electrical property measuring device, configured to measure a needling instrument to obtain the electrical property parameter.

10. A thermal treatment planning method, adapted to a thermal treatment planning system, wherein the thermal treatment planning method comprises:
obtaining a medical condition setting;
performing a curve fitting operation based on a plurality of surgical situation information provided by a surgical situation database and a plurality of setting parameters comprised in the medical condition setting, so as to obtain a surgical evaluation information corresponding to the medical condition setting; and
displaying the surgical evaluation information, and determining whether to optimize the plurality of surgical situation information in the surgical situation database based on the surgical evaluation information according to whether the surgical evaluation information is reliable.

11. The thermal treatment planning method as claimed in claim 10, further comprising:
receiving a confirmation signal from an external source to determine whether the surgical evaluation information is reliable; and
displaying a request to input the surgical condition setting again if the surgical evaluation information is not reliable, and feeding the surgical situation information corresponding to the surgical evaluation information back to the surgical situation database to optimize the surgical situation database if the surgical evaluation information is reliable.

12. The thermal treatment planning method as claimed in claim 10, wherein the surgical evaluation information comprises a temperature distribution information and a thermal damage range information.

13. The thermal treatment planning method as claimed in claim 10, further comprising:
validating the plurality of surgical situation information based on a plurality of experimental validation results to determine whether to correspondingly adjust the plurality of surgical situation information.

14. The thermal treatment planning method as claimed in claim 13, further comprising:
adjusting the curve fitting operation based on the experimental validation results.

15. The thermal treatment planning method as claimed in claim 10, further comprising:
obtaining a plurality of surgical parameters comprising at least one of a tissue profile parameter, a tissue thermal property parameter, a physical parameter of a needling instrument or a biological tissue, and an electrical property parameter; and
setting up the surgical parameter database based on the surgical parameters.

16. The thermal treatment planning method as claimed in claim 15, wherein obtaining the surgical parameters comprises:
capturing a medical image of a tissue of a biological organ; and
analyzing the medical image to obtain the tissue profile parameter.

17. The thermal treatment planning method as claimed in claim 15, wherein obtaining the surgical parameters comprises:
measuring a needling instrument to obtain the property parameter of the needling instrument.

18. The thermal treatment planning method as claimed in claim 15, wherein obtaining the surgical parameters comprises:
measuring a needling instrument to obtain the electrical property parameter.
